(19) United States
(12) Patent Application Publication
(10) Pub. No.: US 2024/0123633 A1
(43) Pub. Date: Apr. 18, 2024

(54) SURGICAL ROBOTIC SYSTEM AND APPLICATIONS AND PARTS THEREFORE

(71) Applicant: MICROSURE B.V., Son (NL)

(72) Inventor: Erik Niels BOERMA, Eindhoven (NL)

(21) Appl. No.: 18/555,679

(22) PCT Filed: Apr. 21, 2022
(86) PCT No.: PCT/EP2022/060568

§ 371 Date: Oct. 16, 2023

(30) Foreign Application Priority Data
May 4, 2021 (EP) 21172106.3

(51) Int. Cl. B25J 15/08; A61B 17/3201; A61B 34/30; A61B 90/00

(52) U.S. Cl. CPC B25J 15/08; A61B 17/3201; A61B 34/30; A61B 90/08; A61B 46/10

(57) ABSTRACT

The present invention relates to an end-effector for a surgical, microsurgical or super-microsurgical robot arm, wherein the end-effector at least comprises: an end-effector base, and a first movable finger and a second movable finger extending from the end-effector base, the first and second movable fingers being configured and adapted to hold and manipulate at least one surgical instrument.

The present invention belongs to the technical field of robots for use in surgery, microsurgery or super-microsurgery procedures. In particular, the present invention relates to an end-effector, end-effector system and a robot arm including said end-effector system for use in surgery, microsurgery or super-microsurgery.

In particular, the end-effector, end-effector system and robot arm can be used in performing anastomosis.

Different kinds of surgical instruments are usually necessary during surgery, microsurgery or super-microsurgery procedures.

Said surgical instruments can be configured to be manually held and manipulated by an operator (e.g., a surgeon) during surgery.

Alternatively, said surgical instruments may be adapted to be robotically manipulated.

Robotic manipulator interfaces for use in a surgical (or even microsurgical) robot are known in the art.

EP3363401A1 discloses a robotic manipulator interface for coupling a hinged surgical tool to a manipulator of a surgical robot, the interface comprising a first interface member to be coupled to the manipulator, and a second interface member to be coupled to the first interface member, the second interface member being arranged to mount the hinged surgical tool, wherein the first interface member comprises a pinching mechanism for pinching the hinged surgical tool when the second interface member is coupled to the first interface member.

EP1232836A1 discloses a robot carrier for tweezers, the robot carrier comprising a housing to be mounted on the arm of the robot and enclosing an operating drive for tweezers and a mounting and indexing support. A motor actuates pusher cam arms to engage the legs of the tweezers to open and close them. The housing is open at one end to receive the tweezers.

Surgical instruments of the same kind, even from the same manufacturer, may have some small structural differences between one another, especially when hand-finished or when some slight constructional changes are carried out by the manufacturer.

In these cases, known end-effectors may be unable to reliably interface with the slightly modified surgical instrument. Additionally, a significant number of instruments for use in surgical, microsurgical or super-microsurgical procedures are specifically designed for manual use.

These manual instruments poorly interface with known end-effectors, and thus need to be manually operated.

Another limit of said known solutions is that it is often difficult to maintain a sterile barrier during surgical instrument replacement.

Since surgical instrument replacement is carried out over an open wound of the patient, it is fundamental to maintain the sterile barrier to prevent the risk of infections or other diseases for the patient subject to surgery.

Yet another limit of said known solutions is that they do not always allow the operator to easily and safely perform surgical instrument replacement.

In particular, there is the risk of accidental interaction with the patient and/or with other devices or objects that are present in the surroundings.

Another issue of said known solutions relates to the risk of unwanted interference with medical gloves worn by the operator.

In the light of the above, it is an object of the present invention to provide an improved end-effector and an end-effector system, inter alia wherein the end-effector and the end-effector system have an improved adaptability, and capable of reliably interfacing even with surgical instruments that are designed for manual operation, further to provide an end-effector and an end-effector system allowing to perform surgical instrument replacement while maintaining the sterile barrier, thereby preventing the risk of infections or other diseases for the patient and to provide an end-effector and an end-effector system allowing the operator to easily and safely perform surgical instrument replacement, without the risk of undesired interaction with the patient and/or other devices or objects that are present in the surroundings and/or interference with medical gloves worn by the operator.

The above-specified objects are achieved by the provision of an end-effector according to claim 1 and an end-effector system according to claim 3.

Accordingly, an end-effector for a surgical, microsurgical or super-surgical robot arm is provided, wherein the end-effector at least comprises:
an end-effector base; and
at least a first movable finger and at least a second movable finger extending from the end-effector base, wherein the first and second movable fingers are configured and adapted to hold and manipulate at least one surgical instrument.

The concept of this end-effector according to the present disclosure and invention is inter alia but not limited by this based on the idea that the end-effector is capable to hold and manipulate a surgical instrument, even when the surgical instrument is not adapted to the end-effector. Further, the end-effector shall be capable to hold the surgical instrument directly or indirectly, as it should also be capable to work with a sterile drape so that cleaning and sterilizing of the end-effector is easier and faster. By this, inter alia the object is addressed that surgical instruments are mainly designed for manual use but not for use with a robot. So, manual instruments are available but interface poorly with existing end-effectors of surgical robots. Usually, a surgical robot is mounted with specific robot targeted instruments. These however are not readily available. With the end-effector as disclosed herein, it is possible to use surgical instruments that have a track record of being suitable for the intended surgery. In particular, the surgical instrument must not be adapted to fit with the end-effector, rather, the end-effector is capable to work with any surgical tool just as the hand of the surgeon would be capable to do.

In other words:

The present disclosure provides an end-effector.

The end-effector is configured and adapted for use in a surgical, microsurgical or super-microsurgical robot arm.

In particular, the robot arm may be configured and adapted to perform anastomoses.

The end-effector includes an end-effector base.

The end-effector further includes a first movable finger and a second movable finger.

The first and second movable fingers extend from the end-effector base. The first and second movable fingers are configured and adapted to hold and manipulate at least one surgical instrument.

The first and second movable fingers of the end-effector may be hollow bodies.

Accordingly, the overall weight of the end-effector can be significantly reduced.

In addition, the space defined within the hollow bodies may be used for housing additional components of the end-effector. Accordingly, one or more additional components can be provided without increasing the overall size of the end-effector.

The present invention further provides an end-effector system. Accordingly, an end-effector system for a surgical, microsurgical or super-microsurgical robot arm is provided, wherein the end-effector system at least comprises:
the end-effector as described herein, especially as above;
at least one sterile drape configured to cover the end-effector, and
at least one surgical instrument adapter which is configured to slide onto the draped first and second fingers of the end-effector and be locked in place by means of a locking mechanism,
wherein the surgical instrument adapter includes a connection element for connection to at least one surgical instrument to hold the at least one surgical instrument in a predefined orientation with respect to the end-effector, and
wherein the surgical instrument adapter is configured to transmit a movement of a tip portion of the first and second fingers of the end-effector to the at least one surgical instrument to manipulate the at least one surgical instrument.

The end-effector system is configured and adapted for use in a surgical, microsurgical or super-microsurgical robot arm.

In particular, the surgical, microsurgical or super-microsurgical robot arm may be configured and adapted to perform anastomoses. The end-effector system comprises an end-effector as described above.

Further, the end-effector system comprises a sterile drape configured to cover the end-effector. By this, the handling of the end-effector is facilitated and easier, as cleaning after use is facilitated and also the sterilization is simplified. In particular, the use of sterile drapes allows a faster and easier handling and use of the end-effector.

Still further, the end-effector system comprises a surgical instrument adapter.

In particular, the surgical instrument adapter is configured to slide onto the draped first and second fingers of the end effector and be locked in place by means of a locking mechanism.

The surgical instrument adapter includes a connection means for connection to at least one surgical instrument.

The connection means (e.g. a clip) allows to hold the at least one surgical instrument in a predefined orientation with respect to the end-effector during surgery.

Advantageously, the connection element may include or may be embodied as a clip.

The concept of a clip allows a fixation without any tools.

The clip is part of the adapter. The adapter is in use placed on the fingers of the end-effector. At that time the fingers can be covered by a drape.

The adapter can also be held by form-fit on the fingers or the fingers covered by a drape.

The instrument is placed and held in use in the clip(s).

The clips may allow movement of the instrument. There can be live hinges for this, such that the clips can move relatively to the adapter, still being materially connected with the adapter.

In other words, in use of the system the fingers of the end-effector may be covered with the drape, the adapter may be placed onto the fingers and the drape, and the surgical instrument may be clipped into the clip of the adapter to fix it in the clip and thereby in the end-effector. This way the fixation of the surgical instrument and to the end-effector is safe and easy to use.

In case of surgical instruments that are designed for manual operation, the surgical instrument adapter may be connected to the portion of the surgical instrument that is intended as the human interface of the instrument (i.e., the portion that is designed to be held by the fingers of the operator during surgery).

Alternatively, the surgical instrument adapter may be configured for connection with a different portion of the surgical instrument, other than the one that is intended as the human interface.

The surgical instrument adapter is configured to transmit a movement of a tip portion of the first and second fingers of the end-effector to the at least one surgical instrument to manipulate the at least one surgical instrument.

Thus, the at least one surgical instrument is manipulated by the first and second fingers of the end-effector through the surgical instrument adapter.

The presence of the sterile drape allows to maintain the sterile barrier.

The surgical instrument adapter allows to improve adaptability of the end-effector.

In particular, the surgical instrument adapter allows the end-effector to reliably interface with a respective kind of surgical instrument even in case of sight structural differences (e.g., as a consequence of being hand-finished or when some small constructional changes are carried out by the manufacturer).

Furthermore, the surgical instrument adapter allows the end-effector to reliably interface with surgical instruments that are designed for manual operation.

The surgical instrument adapter further allows the operator to easily and safely perform surgical instrument replacement during the surgical procedure.

In particular, the risk of unwanted interaction with the patient and/or other devices or objects that are present in the surroundings can be largely avoided.

Similarly, the risk of unwanted interference with the operator's gloves, which may result in tearing of the gloves and thus a breach of the sterile barrier, can be prevented.

The invention is based on the basic idea that, by the provision of a surgical instrument adapter as defined above, it is possible to improve adaptability of the end-effector, so that the end effector can reliably interface with a respective kind of surgical instrument even in case of slight structural changes. Further, the surgical instrument adapter allows the end-effector to reliably interface with surgical instruments that are designed for manual operation. Still further, the surgical instrument adapter allows the operator to easily and safely perform surgical instrument replacement in the course of the surgical procedure, thereby avoiding the risk of unwanted interaction with the patient and/or other devices or objects that are present in the surroundings and/or without interference with medical gloves worn by the operator. Additionally, the presence of the sterile drape allows maintaining the sterile barrier over the whole course of the surgical procedure, thereby reducing the risk of infections and/or other diseases for the patient subject to surgery.

Accordingly, the need for surgical instrument replacement in the course of the surgical procedure can be reduced.

The locking mechanism may be configured to switch between:
a first, unlocked state, and
a second, locked state to secure the surgical instrument adapter in place onto the first and second fingers of the end-effector.

With the locking mechanism it is possible that intuitive and easy unlocking and locking of the surgical instrument can be provided. This is necessary to ensure the safety of operation.

When the locking mechanism is brought into the second, locked state, a spring force may generate from the sterile drape as a consequence of the compression exerted by the locking mechanism.

This way it is possible, that the locking mechanism can be stably retained in place. It is an easy and safe-by-design solution to provide the necessary locking force for securing the surgical instrument.

Alternatively, a spring force may generate from the adapter as a consequence of the compression exerted by the locking mechanism being brought into the second, locked state, which cooperates to maintain the locking mechanism in place.

Also here, the effect is that the locking mechanism can be stably retained in place. Also, it is an easy and safe-to-use solution. It can be combined with the foregoing option that the spring force is generated from the sterile drape.

As a further alternative, a spring force may generate from the sterile drape as a consequence of the elongation of the sterile drape determined by the locking mechanism being brought into the second, locked state.

Also here, the effect is that the locking mechanism can be stably retained in place. Also, it is an easy and safe-to-use solution. It can be combined with the foregoing options.

The locking mechanism is part of the adapter and with the adapter the locking mechanism may slide onto the sterile drape and be fixed to the fingers of the end-effector without the risk of producing any particles due to rubbing between the surfaces.

Accordingly, sterility and safety conditions of the surgical site can be further improved.

The locking mechanism may be a bi-stable mechanism having a natural preference for the unlocked state.

This assures that the locking mechanism is always open upon placement, thereby avoiding hampered handling.

In such a case, once positioned onto the draped fingers of the end-effector, the locking mechanism is manually brought into the second, locked state.

Alternatively, the locking mechanism can be a bi-stable mechanism having a natural preference for the locked state.

This ensures that the locking mechanism is automatically locked once placed onto the draped fingers of the end-effector.

Transition between the locked and unlocked state of the locking mechanism may be carried out with the aid of a locking tool. The surgical instrument adapter may be configured to maintain the alignment between the longitudinal axis of the surgical instrument and the rolling axis of the end-effector during surgery.

Alternatively, the surgical instrument adapter may be configured to hold the surgical instrument in a position where the longitudinal axis of the surgical instrument is offset by a predefined angle with respect to the rolling axis of the end-effector during surgery.

Advantageously, the surgical instrument adapter may be configured to hold an additional surgical instrument.

In particular, the additional surgical instrument may include scissors.

It is important for the surgeon that he can use several surgical tools, which are adapted to the current situation of the surgery this is provided and enabled by the end-effector system.

Furthermore, the surgical instrument adapter is configured to maintain the alignment between the longitudinal axis of the at least one surgical instrument and the rolling axis of the end-effector during surgery. This way, the needed accuracy for micro-surgery or super-microsurgery can be ensured.

Also, it is possible that the surgical instrument adapter is configured to hold the at least one surgical instrument in a position where the longitudinal axis of the at least one surgical instrument is offset by a predefined angle with respect to the rolling axis of the end-effector during surgery. This way, more freedom of design is possible, but still the accuracy needed for the surgery can be maintained.

Advantageously, the sterile drape can be a glove-like drape covering the end effector.

This allows a better adaptation of the sterile drape with respect to the shape of the end-effector.

Advantageously, the glove-like drape and the end-effector may have complementary shapes.

Accordingly, undesired rotation or any movement of the glove-like drape and the end-effector with respect to one another can be prevented. Rather, the relative position to each other can be and will be maintained.

The sterile drape may be connected to another sterile drape that covers the robot arm.

For instance, the sterile drape may be a separate part that is temporary or permanently fixed to the sterile drape covering the robot arm.

When connected to the sterile drape covering the robot arm, the sterile drape may however maintain freedom to rotate around the rotational axis of the end-effector.

In particular, the sterile drape may have complete freedom to rotate around the rotational axis of the end-effector.

Alternatively, rotation of the sterile drape around the rotational axis of the end-effector can be limited to a certain maximum angle.

The sterile drape may have a uniform thickness.

This allows obtaining a solution that is more economical and easier to manufacture.

Alternatively, the sterile drape may have local thickness variations.

This allows to obtain an improved configuration of the sterile drape, that allows largely preventing the occurrence of rupture and/or puncturing.

For instance, parts of the sterile drape that are subject to a greater stress can be made thicker.

Further, parts of the sterile drape can be optimized for use as compression or elongation springs.

Still further, the provision of a sterile drape having different thicknesses allows achieving an enhanced compliance with the end-effector.

The sterile drape may include a sealing interface.

In particular, the sealing interface may be configured for connection with respect to other parts of the surgical, microsurgical or super-microsurgical robot arm.

Alternatively, the sterile interface may be configured for connection with respect to the sterile drape covering the surgical, microsurgical or super-microsurgical robot arm.

In particular, the sealing interface may be in the shape of a circular lip.

Further, the sealing interface can be embodied such that it is or has a circumferential lip for sealing.

The sterile drape can be made of a thermally insulating material.

In the course of a surgical procedure, the surface temperature of the end-effector and the surgical instrument must remain below 43°.

The provision of a thermally-insulated sterile drape allows more easily maintaining the desired temperature below 43°, especially when compared to sterile bags that are used in the art.

Additionally or alternatively, the sterile drape can be made of an electrically insulating material.

During the surgical procedure, it is important to shield the patient from harmful electrical current.

The provision of an electrically-insulating sterile drape allows obtaining improved electrical insulation and thus a better protection for the patient, especially when compared to the sterile bags that are used in the art.

Advantageously, the end-effector system may further include indicating elements for indicating whether the locking means is in the first, unlocked state or the second, locked state.

This allows to intuitively provide the operator with a feedback on the correct positioning and orientation of the adapter on the fingers of the end-effector, thereby preventing the occurrence of errors.

In particular, the indicating element may include an alignment feature that visually indicates whether the locking element is in the first, unlocked state or the second, locked state by means of alignment or misalignment between a surface of the alignment element and a surface of the surgical instrument adapter.

Additionally or alternatively, the indicating element may include means for emitting a visual and/or acoustic signal to the operator.

Finally, the present invention provides a surgical, microsurgical or super-microsurgical robot arm including the above-described end-effector system.

In particular, the robot arm may be configured and adapted to perform anastomoses.

Further advantages of the present invention shall now be disclosed in connection with the drawings, where:

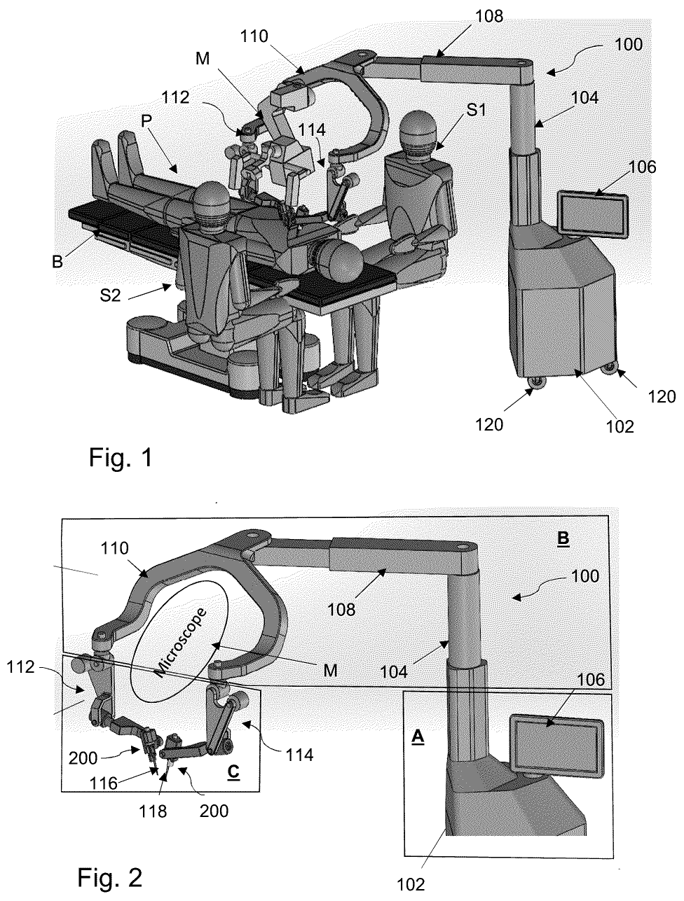

FIG. 1 is a schematic perspective view of a surgical robotic system during a surgical operation in an operation room;

FIG. 2 is a perspective view of a detail of the surgical robotic system of FIG. 1, where different sections of the surgical robotic system are identified;

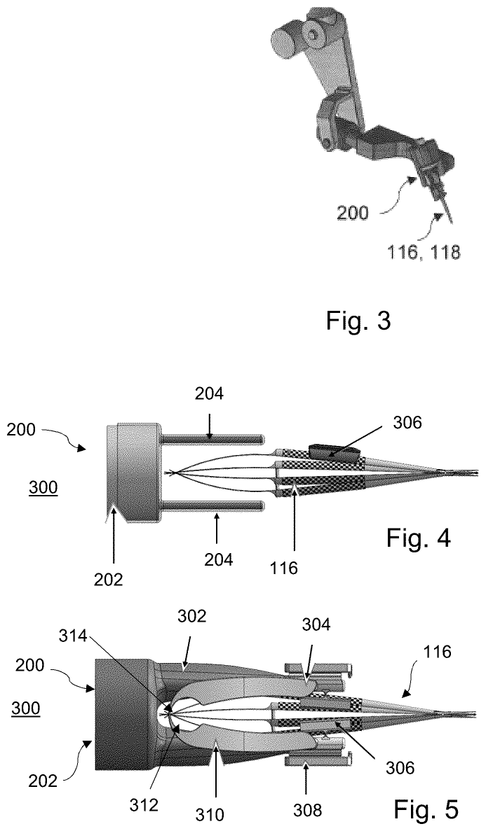

FIG. 3 is a perspective view showing a detail of an end effector of the surgical robotic system of FIG. 1;

FIG. 4 is a plan view of an end-effector for a surgical, microsurgical or super-microsurgical robot arm according to an embodiment of the present invention;

FIG. 5 is a plan view of an end-effector system according to an embodiment of the present invention, the system including the end-effector of FIG. 4, a sterile drape and a surgical instrument adapter,

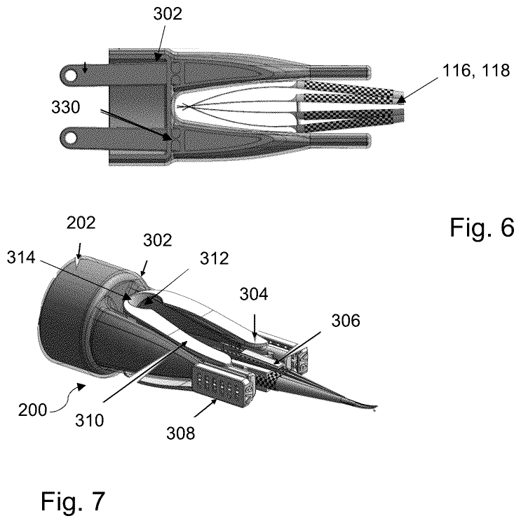

FIG. 6 is a cross-section view of the end-effector system of FIG. 5;

FIG. 7 is a perspective view of the end-effector system of FIG. 5;

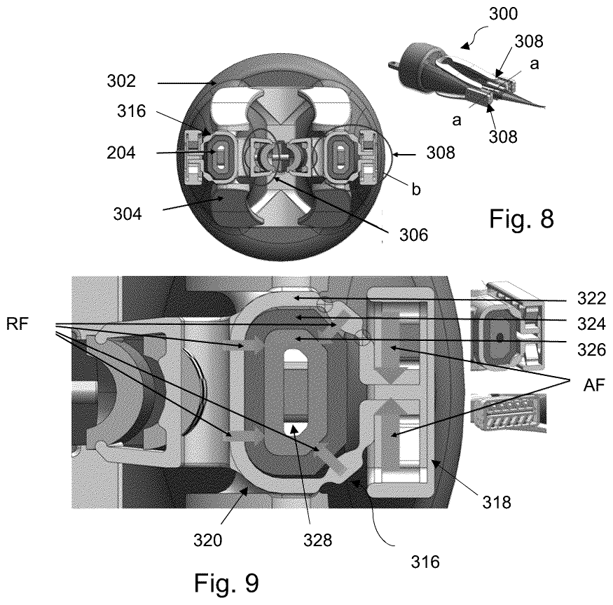

FIG. 8 is a cross section view taken along line a-a, and

FIG. 9 is an enlarged view of detail b of FIG. 8.

FIG. 1 shows a perspective view of a surgical robotic system 100 during operation and applications and parts thereof.

Here, a first surgeon S1 and a second surgeon S2 are located at a respective side of a bed B to perform a surgical operation on a patient P, laying on the bed B.

The surgical robotic system 100 includes a base station 102.

The surgical robotic system 100 further includes a base column 104.

The base column 104 is in the shown embodiment a cylindrical pillar.

The base station 102 includes a display module 106 for displaying information to one or more operators such as the surgeons S1, S2 or other personnel.

Also, the display module 106 may be used to provide a user interface allowing one or more operators to insert a user input.

Further, the surgical robotic system 100 is equipped in the shown embodiment with a suspension arm 108, a fork-like element 110, a first surgical, microsurgical or super-microsurgical robot arm 112, a second surgical, a microsurgical or super-microsurgical robot arm 114, a first surgical instrument 116 and a second surgical instrument 118. Other possible embodiment may be equipped with a different number of robot arms 112, 114, i.e. one or more robot arms, or one or more surgical instruments 116, 118.

The base station 102 is provided with wheels 120 (two shown in FIG. 1). This allows easy placement of the surgical robotic system 100 at a desired location within the operating room.

The suspension arm 108 is in the shown embodiment horizontally arranged. It is also adjustable in its length, as it has two parts with provide the possibility to adjust the length by telescopically moving the parts of the suspension arm 108 relatively to each other. It is also possible that other mechanism for length adjustment can be used, e.g. SCARA-mechanism, hinge solutions and the like.

The suspension arm 108 is connected to the base column 104.

The suspension arm 108 carries the fork-like element 110 (bracket 110).

Here, the fork-like element 110 carries the first surgical, microsurgical or super-microsurgical robot arm 112 and the second surgical, microsurgical or super-microsurgical robot arm 114 (FIG. 2).

Each of the first and second surgical, microsurgical or super-microsurgical robot arms 112, 114 is connected to one end-effector system carrying a surgical instrument 116, 118.

The end-effector system may be the end-effector system 200 described below.

The system 100 may as well be used in combination with the end-effector system 300 described below.

The surgical instruments 116, 118 may be of the same kind or be different from each other.

The suspension arm 108 is configured to rotate about the longitudinal axis of the base column 104 to allow desired positioning of the fork-like element 110 and the robot arms 112, 114 with respect to the surgical site.

Similarly, the fork-like element 110 is configured to rotate about an axis of the suspension arm 108, i.e. parallel to the longitudinal axis of the base column 104.

In the shown configuration, the surgical robotic system 100 is adapted for use with a conventional microscope M (FIG. 1 and FIG. 2).

Alternatively, a fully digital microscope or a so-called hybrid microscope (not shown in FIG. 1) can as well be used. In such a case, one or more surgeons will be located at a separate console, and a robot trolley will be located at the position of one of the surgeons S1, S2 of FIG. 1.

The surgical robotic system 100 aims to provide one large patient side setup, this meaning that no assembly is required after draping.

FIG. 2 shows different sections A, B, C of the surgical robotic system 100.

In section A, there is no movement and everything is stationary.

The display means 106 (included in section A) can be activated by one or more operators.

In section B, there is some movement during the surgery, in order to correctly reposition the surgical instruments 116, 118 over the patient P.

As shown in FIG. 2, a space is defined between the surgical robotic arms 112, 114 for a microscope (not illustrated but only symbolized in FIG. 2).

Section C includes, inter alia, the end-effectors (such as the end-effector 200 described below). Here, several movements occur in the course of the surgical procedure.

A detail of one of the end-effectors 200, carrying a respective surgical instrument 116, 118 is shown in FIG. 3.

The functions of the end-effector are, inter alia, to actuate the surgical instruments 116, 118, to provide a mounting interface and rotate around the instrument axis.

FIG. 4 shows a plan view of an end-effector 200 for a surgical, microsurgical or super-microsurgical robot arm, according to an embodiment of the present invention.

The end-effector 200 may be used in combination with the surgical robotic system 100 of FIG. 1 and FIG. 2.

In such a case, a respective end effector 200 will be connected to each of the surgical, microsurgical or super-microsurgical robot arms 112, 114 (FIG. 2).

The end-effector 200 includes an end-effector base 202.

The end-effector 200 further includes a first movable finger 204 and a second movable finger 204, extending from the end-effector base 202.

In particular, the first and second movable fingers 204 of the end-effector 200 are configured and adapted to hold the at least one surgical instrument 116, 118 and by means of movement of the robot arm, six degrees of movement of the surgical instrument 116, 118 can be facilitated. Additionally and not included in the six degrees of freedom mentioned is the open-close movement of the instrument, which is also provided.

In the shown embodiment, the first and second movable finger 204 are draped with a drape 302 (FIG. 5).

The draping 302 is a sterile drape as described below. Further, it is adapted to fit on the movable finger 204 and to cover the end-effector 200 completely at least with its open surfaces during use, i.e. to cover all parts which are exposed to the patient and operational site.

Advantageously, the first and second movable fingers 204 may be configured as hollow bodies.

This significantly reducing the overall weight of the end-effector 200.

Additionally, the hollow bodies may be adapted for housing additional components without increasing the overall dimensions of the end-effector 200.

In the present embodiment, the end-effector 200 is made as a single body. This allows to prevent play and assembly risks.

FIG. 5 shows an end effector system 300 according to an embodiment of the present invention.

The end-effector system 300 may be used in combination with the surgical robotic system 100 of FIG. 1 and FIG. 2.

In such a case, a respective end-effector system 300 will be connected to each of the surgical, microsurgical or super-microsurgical robot arms 112, 114.

The end-effector system 300 includes the end-effector 200 described above with reference to FIG. 5.

Further, the end-effector system 300 includes a sterile drape 302 which will be further described in the following.

The sterile drape 302 is configured to cover the end-effector 200 as illustrated in FIG. 5.

Still further, the end-effector system 300 includes a surgical instrument adapter 304.

In particular, the surgical instrument adapter 304 is configured to slide onto the draped first and second fingers 204 of the end-effector 200 and be locked in place by means of a locking mechanism (not shown), which will be further described in the following.

In the present embodiment, the surgical instrument adapter 304 is substantially U-shaped.

A partially-round recess 312 is formed proximal to the curved part 314 of the U-shaped surgical instrument adapter 304.

In the shown embodiment, in the curved part 314 has a reduced thickness than the other parts of the U-shaped surgical instrument adapter 304, resulting in an increased elasticity (FIG. 5 and FIG. 7).

The surgical instrument adapter 304 is designed for a specific kind or family of surgical instruments.

The surgical instrument adapter 304 may be configured for connection with a part of the at least one surgical instrument 116, 118 that is intended as the human interface of the instrument.

The surgical instrument adapter 304 can be provided with a labelling element, such as a print, a QR code, a barcode, and/or RFID code or tag for providing information about the adapter.

Alternatively, the surgical instrument adapter 304 may be configured for connection with parts of the at least one surgical instrument 116, 118 different than the one that is intended as the human interface.

The surgical instrument adapter 304 includes connection means for connection 306, here a clip 306 to the at least one surgical instrument 116, 118 to hold the at least one surgical instrument 116, 118 in a predefined orientation with respect to the end-effector 200.

For instance, the at least one surgical instrument 116, 118 may be positioned and retained in place so that the longitudinal axis of the at least one surgical instrument 116, 118 is aligned with respect to the rolling axis of the end-effector 200.

Alternatively, the at least one surgical instrument 116, 118 may be positioned and retained in place so that a small offset in distance and/or angle exists between the longitudinal axis of the at least one surgical instrument 116, 118 and the rolling axis of the end-effector 200.

As a further alternative, the at least one surgical instrument 116, 118 may be positioned and retained in place so that a large offset exists between the longitudinal axis of the at least one surgical instrument 116, 118 and the rolling axis of the end-effector 200.

In the shown embodiment, the locking mechanism 308 of the surgical instrument adapter 304 includes a moving part, which is gripping with form-fit on the fingers and the drape (FIG. 5, FIG. 7 and FIG. 8).

The structure of the locking mechanism 308 according to the present embodiment is shown in detail in the cross-section views provided in FIG. 8 and FIG. 9.

In the present embodiment, the locking 308 includes two moving arms 316 having a first arm portion 318 and a second arm portion 320, internal with respect to the first arm portion 318. The second arm portion 320 includes an external layer 322 made of plastic, an intermediate layer 324 made of silicon and an inner layer 326 made of metal. The inner layer 322 defines a void 328 (empty or unfilled space 328).

When the locking mechanism 308 are placed into their operative position to connect the instrument adapter 304 to the at least one surgical instrument 116, 118, the actuating forces AF act as illustrated in FIG. 9 (see also description below) and create the resulting forces RF, which lock the surgical instrument 116, 118. Accordingly, the at least one surgical instrument 116, 118 is stably retained in place at a desired position with respect to the end-effector 200.

The instrument adapter 304 has an instrument clip 306, which holds the surgical instrument 116, 118 and which by means of a compliant connection, here e.g. a live hinge, capable to move relatively to the instrument adapter 304.

The surgical instrument adapter 304 is configured to transmit a movement of a tip portion of the first and second fingers 204 of the end-effector 200 to the at least one surgical instrument 116, 118, connected to the end-effector 200, such that the at least one surgical instrument 116, 118 can be moved and so a movement of the at least one surgical instrument 116, 118 at six degrees of freedom plus the open-close movement of the instrument is facilitated.

In the shown embodiment, a space is defined between the intersection point between the proximal end of the surgical instrument 116, 118 and the instrument adapter 304 and the end-effector base 202 (FIG. 4 and FIG. 5).

Otherwise stated, said intersection point is not in contact with the end-effector base 202.

The locking mechanism 308 is configured to switch between:
a first, unlocked state, and
a second, locked state.

In the second, locked state the locking mechanism secures the surgical instrument adapter 304 in place onto the first and second fingers 204 of the end-effector 200.

In the end-effector system 300 a spring force may generate from the sterile drape 302 as a consequence of the compression exerted by the locking mechanism being brought into the second, locked state.

This facilitates to maintain the locking mechanism in place.

Alternatively, a spring force may generate from the adapter 304 as a consequence of the compression exerted by the locking mechanism being brought into the second, locked state.

This facilitates to maintain the locking mechanism in place.

As a further alternative, a spring force may generate from the sterile drape 302 as a consequence of the elongation of the sterile drape 302 determined by the locking mechanism being brought into the second, locked state.

This facilitates to maintain the locking mechanism in place.

In the shown embodiment, the end-effector system 300 has live hinges 330 (FIG. 6). The live hinges 330 result from the fact that a 3D printed shell as chassis is used. It is a single part with the design intention that the there is no stick-slip effect in the hinge and that there is no hysteresis in the movement of the hinge. Further, there is no machining and the shell is a “one-part-shell”. The hinges are part of the overall from a result from parts with reduced wall thickness, which allow movement at exactly this part of the shell, thereby forming a hinge.

The locking mechanism may be a bi-stable system having a natural preference for the unlocked state. In such a case, once the locking mechanism is placed onto the draped end-effector 200, a lock must be manually activated to bring the locking mechanism into the locked state. This solution has the advantage of ensuring that the locking mechanism is maintained in the unlocked state during placement, thereby preventing hampered handling.

Alternatively, the locking mechanism may be a bi-stable system having a natural preference for the locked state. This ensures that the locking mechanism is automatically locked after placement.

Transition between the locked and unlocked state of the locking mechanism may be carried out with the aid of a locking tool.

Advantageously, the surgical instrument adapter 304 may be configured to hold an additional surgical instrument.

For instance, scissors may be added to any other surgical instrument so as to prevent the need of changing instruments, thereby reducing the overall time needed to perform full anastomoses.

The surgical instrument adapter 304 is configured to maintain the alignment between the longitudinal axis of the at least one surgical instrument 116, 118 and the rolling axis of the end-effector 200 during surgery.

The surgical instrument adapter 304 may be configured to hold the at least one surgical instrument 116, 118 in a position where the longitudinal axis of the at least one surgical instrument 116, 118 is offset by a predefined angle with respect to the rolling axis of the end-effector 200 during surgery.

In the present embodiment, the sterile drape 302 is a glove-like drape 302 covering the end effector 200 (FIGS. 5-7).

This allows a better adaptation of the sterile drape 302 with respect to the shape of the end-effector 200.

In particular, in the shown embodiment, the glove-like drape 302 and the end-effector 200 have complementary shapes (FIG. 5).

This has the advantage that undesired rotation of the sterile drape 302 and the end-effector 200 with respect to one another can be prevented.

The sterile drape 302 may have a uniform thickness.

A sterile drape 302 having uniform thickness has the advantage of being easier and less expensive to manufacture.

Alternatively, the sterile drape 302 may have local thickness variations.

Although more expensive to manufacture, nevertheless this solution has the advantage of largely preventing the occurrence of rupture and/or puncturing.

The sterile drape 302 may be provided with a sealing interface (not shown).

The sealing interface may be configured for connection with respect to other parts of the surgical, microsurgical or super-microsurgical robot arm.

Alternatively, the sealing interface may be configured for connection with respect to the sterile drape 302 covering the surgical, microsurgical or super-microsurgical robot arm.

Preferably, the sealing interface includes a circular lip.

Advantageously, the sterile drape 302 can be made of a thermally insulating material.

In the course of a surgical, microsurgical or super-microsurgical operation, it is required that the surface temperature of the end-effector 200 and surgical instrument 116, 118 are maintained at a temperature below 43° C.

The use of a sterile drape 302 made of a thermally insulating material allows more easily maintaining the surface temperature of the end-effector 200 and surgical instrument 116, 118 at an appropriate temperature below 43° C. when compared to the thin, bag-like drapes of the prior art.

Additionally or alternatively, the sterile drape 302 can be made of a material which is electrically insulating.

This allows to easily and effectively shield the patient P from harmful electrical current, at the same time without the risk of breaking the sterile barrier.

The sterile drape 302 may be a fixed assembly with other sterile drape parts (not shown) that cover the whole surgical robot. In this case, a single sterile drape covers the whole robot arm.

In particular, the glove-like drape 302 can be permanently connected to the sterile drape covering the robot arm (not shown). The glove-like drape 302 is free to rotate around the rotational axis of the end-effector 200 to a maximum angle or even endlessly.

Alternatively, the sterile drape 302 may be separate from the other sterile drape parts that cover the whole surgical robot.

The end-effector 200 may further include indicating means for indicating whether the locking means is in the first, unlocked state or the second, locked state.

In the present embodiment, the indicating means includes an alignment element 310 that visually indicates whether the locking means is in the first, unlocked state or the second, locked state by means of alignment or misalignment between a surface of the alignment element 310 and a surface of the surgical instrument adapter 304.

Additionally or alternatively, the indicating means may include (not shown) means for emitting a visual and/or acoustic signal to the operator.

### REFERENCE LIST

100 Surgical robotic system
102 Base station
104 Base column
106 Display module
108 Suspension arm
110 Fork-like element
112 (First) surgical, microsurgical or super-microsurgical robot arm
114 (Second) surgical, microsurgical or super-microsurgical robot arm
116 Surgical instrument
118 Surgical instrument
120 Wheels
200 End-effector
202 End-effector base
204 Movable finger
300 End-effector system
302 Sterile drape
304 Surgical instrument adapter
306 Instrument clip
308 Means for connection (locking mechanism)
310 U-shaped alignment element
312 Recess
314 Curved part of the surgical instrument adapter
316 Clip arm
318 First clip arm portion
320 Second clip arm portion
322 External layer of the second clip arm portion (plastic)
324 Intermediate layer of the second clip arm portion (silicon)
326 Internal layer of the second clip arm portion (metal)
328 Void/empty space
330 Live hinges
B Bed
M Microscope
S1 First surgeon
S2 Second surgeon
P Patient
AF Actuation forces
RF Resultant forces

1. An end-effector for a surgical, microsurgical or super-microsurgical robot arm, wherein the end-effector at least comprises:
an end-effector base, and
a first movable finger and a second movable finger extending from the end-effector base, the first and second movable fingers being configured and adapted to hold and manipulate at least one surgical instrument.

2. The end-effector according to claim 1,
wherein
the first and second movable fingers of the end-effector are configured as hollow bodies.

3. An end-effector system for a surgical, microsurgical or super-microsurgical robot arm, wherein the end-effector system at least comprises:
the end-effector according to claim 1;
a sterile drape configured to cover the end-effector, and
a surgical instrument adapter which is configured to slide onto the draped first and second movable fingers of the end-effector and be locked in place by means of a locking mechanism,
wherein the surgical instrument adapter includes a clip to at least one surgical instrument to hold the at least one surgical instrument in a predefined orientation with respect to the end-effector, and
wherein the surgical instrument adapter is configured to transmit a movement of a tip portion of the first and second movable fingers of the end-effector to the at least one surgical instrument to manipulate the at least one surgical instrument.

4. The end-effector system of claim 3,
wherein
the locking mechanism is configured to switch between
a first, unlocked state, and
a second, locked state to secure the surgical instrument adapter in place onto the first and second movable fingers of the end-effector.

5. The end-effector system of claim 4,
wherein
a spring force generates from the sterile drape as a consequence of compression exerted by the locking mechanism being brought into the second, locked state, which cooperates to maintain the locking mechanism in place.

6. The end-effector system of claim 4,
wherein
a spring force generates from the surgical instrument adapter as a consequence of compression exerted by the locking mechanism being brought into the second, locked state, which cooperates to maintain the locking mechanism in place.

7. The end-effector system of claim 4,
wherein
wherein a spring force generates from the sterile drape as a consequence of elongation of the sterile drape determined by the locking mechanism being brought into the second, locked state, which cooperates to maintain the locking mechanism in place.

8. The end-effector system according to claim 3,
wherein
the surgical instrument adapter is configured to hold an additional surgical instrument.

9. The end-effector system according to claim 3,
wherein
the surgical instrument adapter is configured to maintain alignment between a longitudinal axis of the at least one surgical instrument and a rolling axis of the end-effector during surgery.

10. The end-effector system according to claim 9,
wherein
the surgical instrument adapter is configured to hold the at least one surgical instrument in a position where the longitudinal axis of the at least one surgical instrument is offset by a predefined angle with respect to the rolling axis of the end-effector during surgery.

11. The end-effector system according to claim 3,
wherein
the sterile drape includes a sealing interface configured for connection with respect to other parts of the robot arm or with respect to the sterile drape covering the robot arm.

12. The end-effector system according to claim 4,
wherein
the end-effector system further includes an indicating element for indicating whether the locking mechanism is in the first, unlocked state or the second, locked state.

13. The end-effector system according to claim 12,
wherein
the indicating element includes an alignment element that visually indicates whether the locking mechanism is in the first, unlocked state or the second, locked state by means of alignment or misalignment between a surface of the alignment element and a surface of the surgical instrument adapter and/or wherein the indicating element includes means for emitting a visual and/or acoustic signal to an operator.

14. A surgical, microsurgical or super-microsurgical robot arm including the end-effector system according to claim 3.

15. The end-effector according to claim 2,
wherein
said hollow bodies are configured and adapted for housing additional components of the end-effector.

16. The end-effector system according to claim 8,
wherein
said additional surgical instrument includes scissors.

17. The end-effector system according to claim 11,
wherein
the sealing interface includes a circular or circumferential lip.
